(19) United States
(12) Patent Application Publication
(10) Pub. No.: US 2008/0051908 A1
(43) Pub. Date: Feb. 28, 2008

(54) SYSTEM AND METHOD FOR ADJUSTING THE THICKNESS OF A PROSTHESIS

(76) Inventors: Laurent Angibaud, Gainesville, FL (US); Alan Seidel, High Springs, FL (US)

Correspondence Address: GREENBERG TRAURIG, LLP, MET LIFE BUILDING, 200 PARK AVENUE, NEW YORK, NY, 10166, US

(21) Appl. No.: 11/843,198

(22) Filed: Aug. 22, 2007

Related U.S. Application Data
(60) Provisional application No. 60/823,186, filed on Aug. 22, 2006.

(51) Int. Cl. A61F 2/38

(52) U.S. Cl. 623/20.32; 623/20.34

(57) ABSTRACT

The present invention relates to a system and method for adjusting a thickness of a prosthesis (e.g., a knee implant). In one embodiment, a knee prosthesis for implantation in a patient is provided, comprising: a tibial tray, wherein the tibial tray comprises an upper surface and a lower surface, and the lower surface of the tibial tray is disposed adjacent a tibia of the patient; a tibial insert, wherein the tibial insert comprises an upper surface and a lower surface; a tibial spacer, wherein the tibial spacer comprises an upper surface and a lower surface, and wherein the tibial spacer is disposed between the tibial tray and the tibial insert such that the lower surface of the tibial spacer is adjacent the upper surface of the tibial tray and the upper surface of the tibial spacer is adjacent the lower surface of the tibial insert; and at least one locking mechanism, wherein the locking mechanism locks the tibial spacer to the tibial tray to form a locked spacer/tray assembly such that the locking mechanism substantially prohibits at least relative up-down movement between the tibial spacer and the tibial tray when the locked spacer/tray assembly is implanted in the patient. In one example, the locking mechanism may further lock the tibial spacer to the tibial insert to form a locked insert/spacer/tray assembly such that the locking mechanism substantially prohibits at least relative up-down movement between the tibial spacer and the tibial insert when the locked insert/spacer/tray assembly is implanted in the patient. In another embodiment, a method for implanting a knee prosthesis in a patient is provided, comprising: providing a tibial tray, wherein the tibial tray comprises an upper surface and a lower surface, and the lower surface of the tibial tray is disposed adjacent a tibia of the patient; providing a tibial insert which has a shelf life and which is not readily re-sterilizable after the shelf life has expired, wherein the tibial insert comprises an upper surface and a lower surface; and providing a tibial spacer which has a shelf life and which is re-sterilizable after the shelf life has expired, wherein the tibial spacer comprises an upper surface and a lower surface, and wherein the tibial spacer is disposed between the tibial tray and the tibial insert such that the lower surface of the tibial spacer is adjacent the upper surface of the tibial tray and the upper surface of the tibial spacer is adjacent the lower surface of the tibial insert.

### RELATED APPLICATIONS

This application claims the benefit of U.S. Provisional Application Ser. No. 60/823,186, filed Aug. 22, 2006, which is incorporated herein by reference in its entirety.

### FIELD OF THE INVENTION

The present invention relates to a system and method for adjusting a thickness of a prosthesis (e.g., a knee implant).

In one embodiment, a knee prosthesis for implantation in a patient is provided, comprising: a tibial tray, wherein the tibial tray comprises an upper surface and a lower surface, and the lower surface of the tibial tray is disposed adjacent a tibia of the patient; a tibial insert, wherein the tibial insert comprises an upper surface and a lower surface; a tibial spacer, wherein the tibial spacer comprises an upper surface and a lower surface, and wherein the tibial spacer is disposed between the tibial tray and the tibial insert such that the lower surface of the tibial spacer is adjacent the upper surface of the tibial tray and the upper surface of the tibial spacer is adjacent the lower surface of the tibial insert; and at least one locking mechanism, wherein the locking mechanism locks the tibial spacer to the tibial tray to form a locked spacer/tray assembly such that the locking mechanism substantially prohibits at least relative up-down movement between the tibial spacer and the tibial tray when the locked spacer/tray assembly is implanted in the patient.

In one example, the locking mechanism may further lock the tibial spacer to the tibial insert to form a locked insert/spacer/tray assembly such that the locking mechanism substantially prohibits at least relative up-down movement between the tibial spacer and the tibial insert when the locked insert/spacer/tray assembly is implanted in the patient.

In another embodiment, a method for implanting a knee prosthesis in a patient is provided, comprising: providing a tibial tray, wherein the tibial tray comprises an upper surface and a lower surface, and the lower surface of the tibial tray is disposed adjacent a tibia of the patient; providing a tibial insert which has a shelf life and which is not readily re-sterilizable after the shelf life has expired, wherein the tibial insert comprises an upper surface and a lower surface; and providing a tibial spacer which has a shelf life and which is re-sterilizable after the shelf life has expired, wherein the tibial spacer comprises an upper surface and a lower surface, and wherein the tibial spacer is disposed between the tibial tray and the tibial insert such that the lower surface of the tibial spacer is adjacent the upper surface of the tibial tray and the upper surface of the tibial spacer is adjacent the lower surface of the tibial insert.

For the purposes of describing and claiming the present invention, the term “undercut” is intended to refer to a configuration such as a groove (having both upper and lower defining faces) or to a configuration such as an overhang (having only one defining face).

Further, for the purposes of describing and claiming the present invention, the term “tab” is intended to refer to a configuration having one or more protrusions or fingers or to a configuration having a protruding strip.

### BACKGROUND OF THE INVENTION

On Feb. 26, 2005, during the Knee Society meeting, Dr. Gerard Engh presented a study of the different sources of Ultra High Molecular Weight Polyethylene (UHMWPE) wear. He used a multiple regression analysis to determine the relative contributions of several factors on the wear of nearly 2000 total knee implants and over 440 unicondylar implants. The study concluded that over 50% of wear could be attributed to prolonged UHMWPE shelf aging.

In this regard, thicker tibial inserts are very rarely used. As a result, these implants are usually associated with a long shelf aging date at the time of the surgery, assuming the part is ever used. Consequently, the thicker tibial inserts could be associated with higher wear. According to an Exactech sales report, surgeons use an insert thicker than 15 mm (e.g., 18 mm or thicker) only 4.7% of the time. In addition, when implants reach the shelf age date limit, the company typically exchanges them for fresher ones. Because the implants manufactured from UHMWPE cannot be easily re-sterilized, the returned implants typically represent a loss for the company.

Also, the tibial insert inventory typically doubles at the time of surgery to provide a back up in case problems arise during surgery (the implants are made from polymer and cannot be re-sterilized by steam). Thick tibial inserts (e.g., 18 mm or thicker) could represent a total of 48 boxes at the time of surgery (e.g., 6 sizes×4 thicknesses×2 sets), which is less than one chance in twenty they would be used.

Moreover, a thick tibial insert is often associated with loss of proximal tibia bone. Since the tibial bone narrows abruptly from proximal to distal, a tibial tray much smaller than the normal tibial tray could be needed.

With regard to patent documents, various knee prosthesis systems have been proposed. Examples include: U.S. Patent Publication 2004/0049284, entitled “UNIVERSAL TIBIAL AUGMENT” and published Mar. 11, 2004 in the name of German et al.; U.S. Pat. No. 4,936,853, entitled “MODULAR KNEE PROSTHESIS” and issued Jun. 26, 1990 in the name of Fabian et al.; U.S. Pat. No. 4,944,757, entitled “MODULATOR [SIC] KNEE PROSTHESIS SYSTEM” and issued Jul. 31, 1990 in the name of Martinez et al.; and U.S. Pat. No. 5,702,464, entitled “MODULAR TRIAL TIBIAL INSERT” and issued Dec. 30, 1997 in the name of Lackey et al.

### BRIEF DESCRIPTION OF THE DRAWINGS

FIG. 1 is a perspective view of a knee prosthesis according to an embodiment of the present invention;

FIG. 2 is another perspective view of the knee prosthesis of FIG. 1;

FIG. 3 is another perspective view of the knee prosthesis of FIG. 1;

FIG. 4 is another perspective view of the knee prosthesis of FIG. 1 (in a disassembled state);

FIG. 5 is another perspective view of the knee prosthesis of FIG. 1 (in a disassembled state);

FIG. 6 is another perspective view of the knee prosthesis of FIG. 1 (in a disassembled state);

FIG. 7 is a perspective view of a tibial spacer portion of the knee prosthesis of FIG. 1;

FIG. 8 is another perspective view of the tibial spacer portion of the knee prosthesis of FIG. 1;

FIG. 9 is another perspective view of the tibial spacer portion of the knee prosthesis of FIG. 1;

FIG. 10 is another perspective view of the tibial spacer portion of the knee prosthesis of FIG. 1;

FIG. 11 is a cross-sectional view of the tibial spacer portion of the knee prosthesis of FIG. 1;

FIG. 12 is another cross-sectional view of the tibial spacer portion of the knee prosthesis of FIG. 1;

FIG. 13 is a perspective view of a tibial spacer portion of a knee prosthesis according to another embodiment of the present invention;

FIG. 14 is another perspective view of the tibial spacer portion of FIG. 13;

FIG. 15 is a perspective view of a tibial spacer portion of a knee prosthesis according to another embodiment of the present invention;

FIG. 16 is another perspective view of the tibial spacer portion of FIG. 15;

FIG. 17 is a perspective view of a tibial spacer portion of a knee prosthesis according to another embodiment of the present invention;

FIG. 18 is another perspective view of the tibial spacer portion of FIG. 17;

FIG. 19 is a perspective view of a tibial spacer portion of a knee prosthesis according to another embodiment of the present invention;

FIG. 20 is another perspective view of the tibial spacer portion of FIG. 19;

FIG. 21 is a diagram which highlights some of the benefits of various embodiments of the present invention as applied to a total knee system;

FIG. 22 illustrates that the moment arm of the resultant load increases with the thickness of the UHMWPE tibial insert; and

FIG. 23 illustrates that a tibial spacer according to an embodiment of the present invention could be “universal” for the entire range of a total knee system.

Among those benefits and improvements that have been disclosed, other objects and advantages of this invention will become apparent from the following description taken in conjunction with the accompanying figures. The figures constitute a part of this specification and include illustrative embodiments of the present invention and illustrate various objects and features thereof.

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention is intended to be illustrative, and not restrictive. Further, the figures are not necessarily to scale, some features may be exaggerated to show details of particular components. Therefore, specific structural and functional details disclosed herein are not to be interpreted as limiting, but merely as a representative basis for teaching one skilled in the art to variously employ the present invention.

In one embodiment, instead of using a thick tibial insert in the case of a large gap between the resected tibia and femur, the composite tibial thickness (i.e. thickness of the tibial tray and tibial insert) may be adjusted using a tibial spacer between the tibial tray and the tibial insert. The tibial spacer may create in essence a variety of thick tibial components that help reduce inventory and promote the use of fresher polyethylene tibial inserts.

In one example (which example is intended to be illustrative and not restrictive), the tibial spacer may be made from biocompatible material (e.g. Titanium, Stainless steel, Cobalt chromium), which could be repackaged and re-sterilized after the shelf life termination date has been reached.

In another example, the tibial spacer may be defined by two faces-one proximal (intended to receive a UHMWPE tibial insert) and one distal (intended to be assembled with a tibial tray). Schematically, the proximal face of the tibial spacer may comprise a geometry relatively similar to the proximal face of the tibial tray, and the distal face of the tibial spacer may comprise a geometry relatively similar to the distal face of the UHMWPE tibial insert. To ensure the tibial spacer completely locks with the tibial tray and/or the tibial insert, a locking screw could be used to expand a locking feature.

In one example, the tibial spacer may be metal and may have a metal male “mushroom” extending therefrom. The male “mushroom” may be segmented to allow it to flex and pass into a female receiving feature in the tibial tray (after the assembly of the tibial spacer to the tibial tray, the “mushroom” may be locked by inserting a screw therethrough in order to disallow deflection of the segmented sections that would lead to disassembly).

In another example, the tibial insert may have extending therefrom a male “mushroom”, fabricated from polyethylene which is more easily elastically deformed (as compared to the metal “mushroom”). The polyethylene “mushroom” may simply be pushed into place in a female receiving feature in the tibial spacer (wherein the male polyethylene “mushroom” deforms elastically, and then recoils to lock itself into place without the need of the locking screw).

In another example, tabs (e.g., anterior and/or posterior) of the tibial spacer, the tibial tray and/or the tibial insert may engage (e.g., slide) under corresponding undercuts (e.g., anterior and/or posterior) of the tibial spacer, the tibial tray and/or the tibial insert to provide added secondary locking.

Referring now to the Figs, various embodiments of the present invention will be discussed.

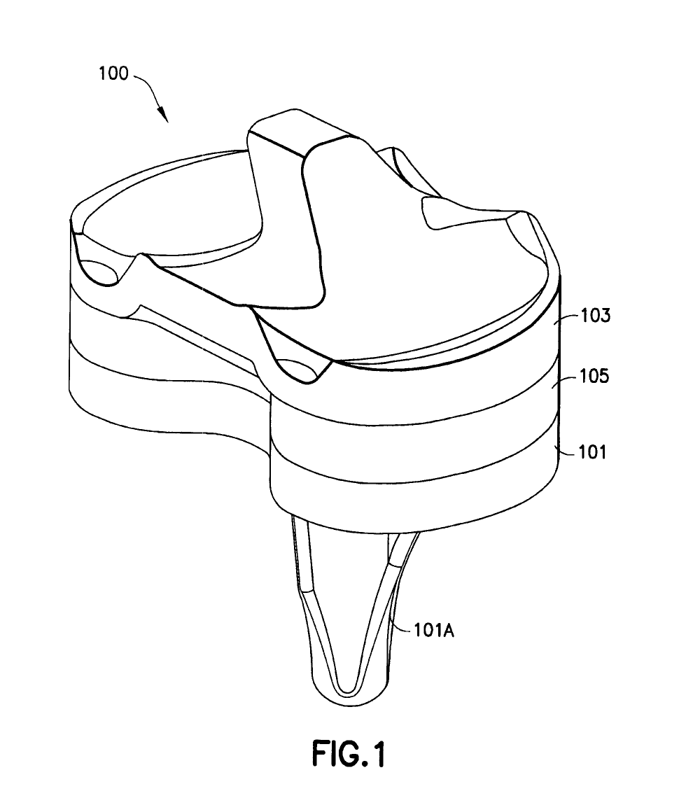

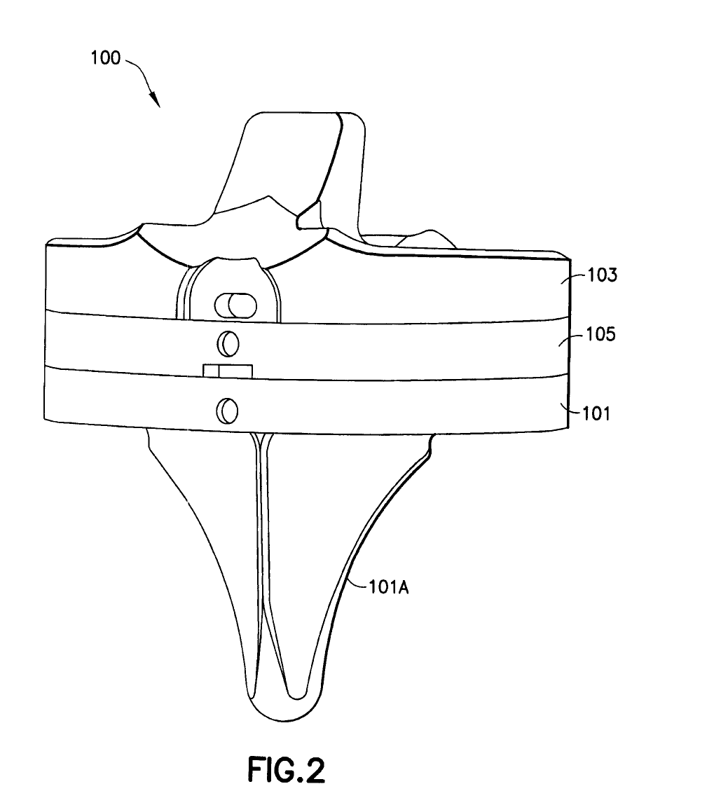

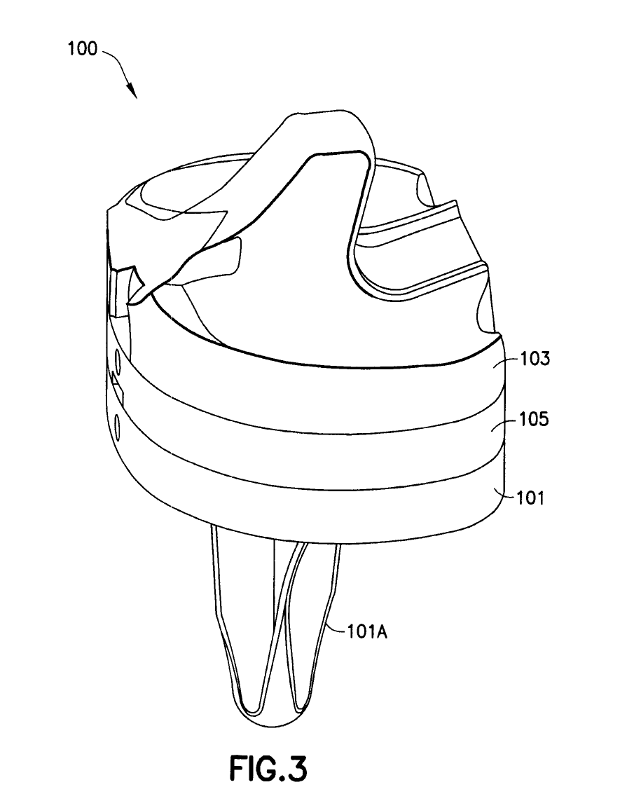

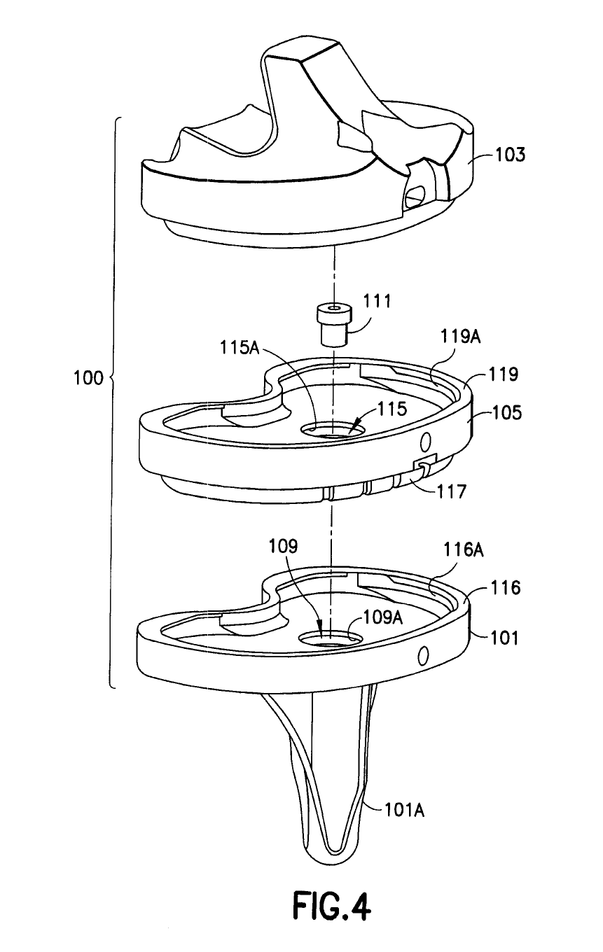

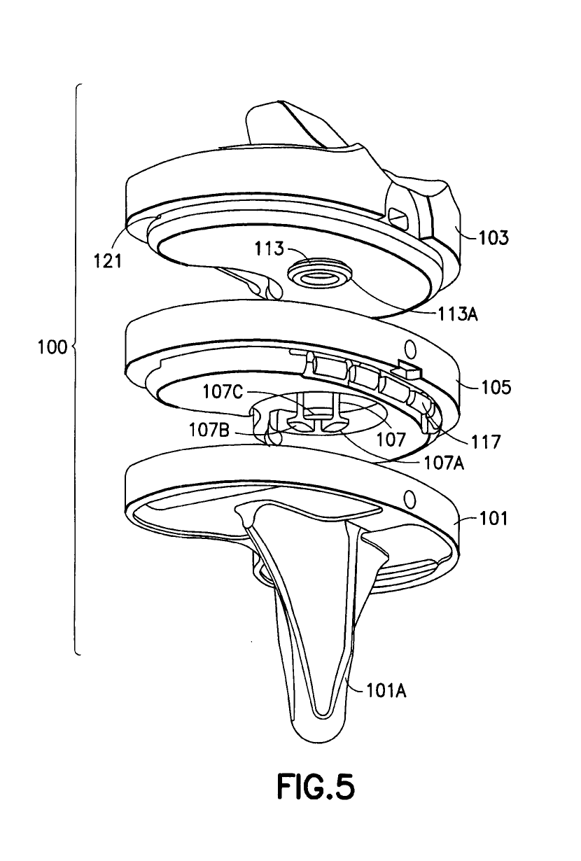

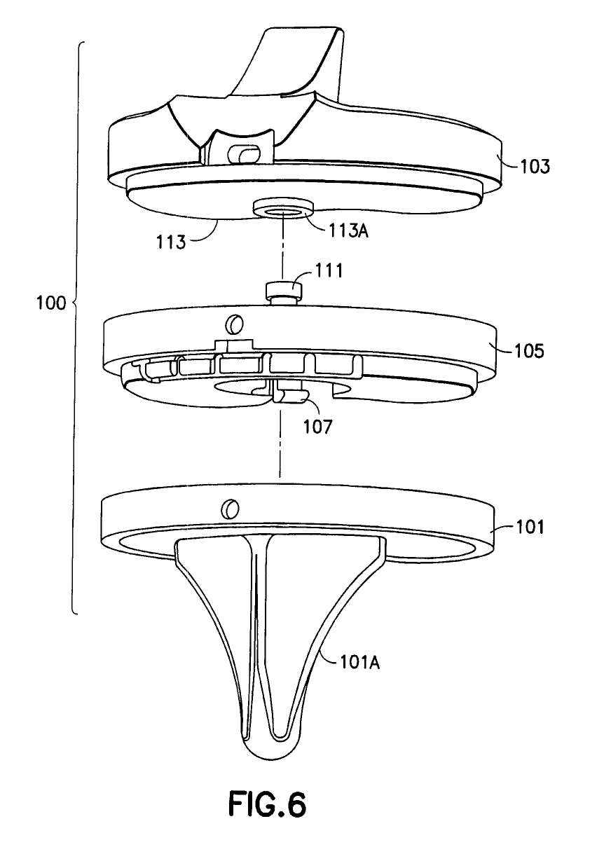

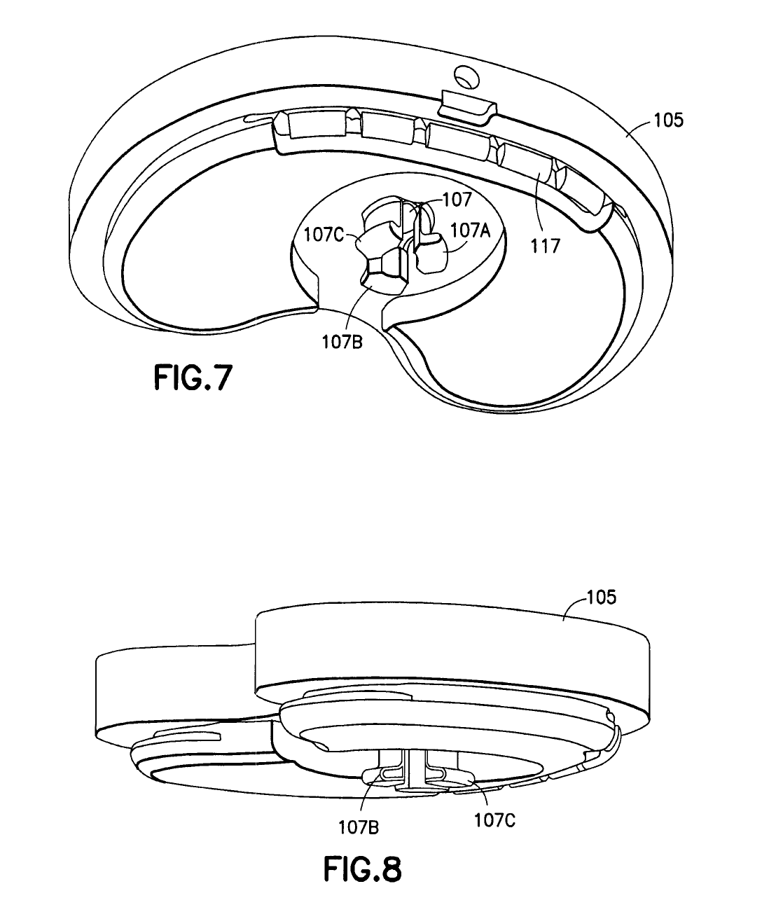

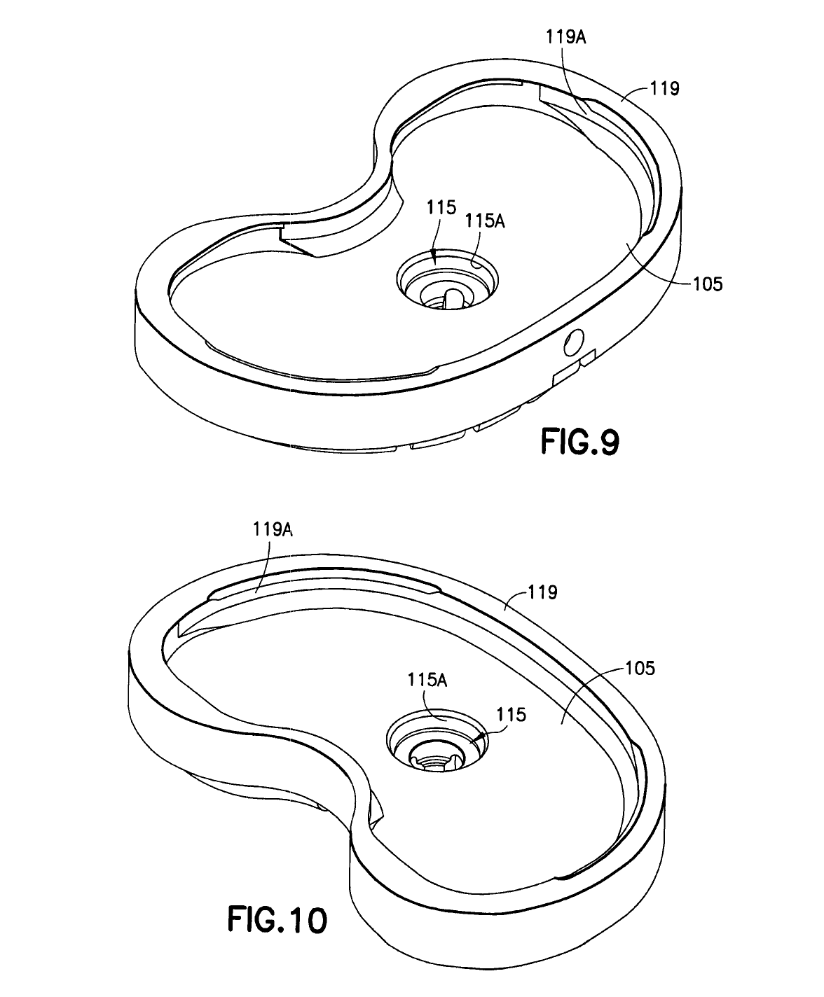

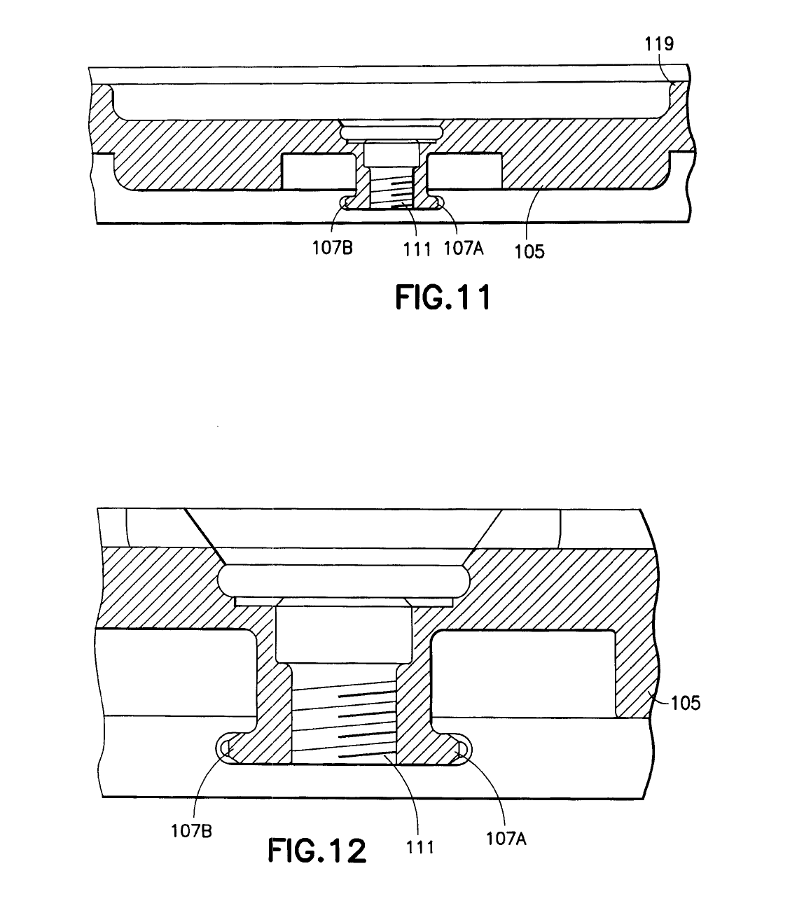

More particularly, with reference first to FIGS. 1-12, it is seen that one embodiment of the present invention provides a knee prosthesis for implantation in a patient. As seen in these Figs., the prosthesis 100 may include tibial tray 101, tibial insert 103 and tibial spacer 105. Tibial tray 101 may comprise an upper surface and a lower surface, and the lower surface of the tibial tray 101 may be disposed adjacent a tibia of the patient (tibial tray 101 may also incorporate tibial tray stem 101A, for implantation into the patient's tibia). Further, tibial insert 103 may comprise an upper surface and a lower surface. Further still, tibial spacer 105 may comprise an upper surface and a lower surface (tibial spacer 105 may be disposed between tibial tray 101 and tibial insert 103 such that the lower surface of tibial spacer 105 is adjacent the upper surface of tibial tray 101 and the upper surface of tibial spacer 105 is adjacent the lower surface of tibial insert 103.

Finally, prosthesis 100 may comprise at least one locking mechanism (discussed in more detail below), wherein the locking mechanism may lock tibial spacer 105 to tibial tray 101 and/or to tibial insert 103 to form a locked assembly such that the locking mechanism substantially prohibits at least relative up-down movement between tibial spacer 105 and tibial tray 101 when the locked assembly is implanted in the patient and wherein the locking mechanism substantially prohibits at least relative up-down movement between tibial spacer 105 and tibial insert 103 when the locked assembly is implanted in the patient.

In one example, the locked assembly may carry joint loads when implanted in the patient.

In another example, the locked assembly allows, when implanted in the patient, ambulation and activities of daily living.

In another example, the upper surface of tibial insert 103 is configured to receive a femoral component which interfaces with a femur of the patient.

In another example, the upper surface and the lower surface of tibial spacer 105 are essentially parallel to one another.

In another example, the upper surface of tibial spacer 105 may be essentially the same shape as the upper surface of tibial tray 101.

In another example, the lower surface of tibial spacer 105 may be essentially the same shape as the lower surface of tibial insert 103.

In another example, the upper surface of tibial spacer 105 may be essentially the same shape as the upper surface of tibial tray 101 and the lower surface of tibial spacer 105 may be essentially the same shape as the lower surface of tibial insert 103.

In another example, the locking mechanism may comprise male feature 107 extending beyond the lower surface of tibial spacer 105 and female feature 109 disposed in the upper surface of tibial tray 101 for receiving male feature 107. Of note, male feature 107 may be deformable in order to lock at least one outwardly extending projection 107A-C of male feature 107 into at least one undercut 109A in female feature 109 (of course, while three outwardly extending projections 107A-C forming a “segmented mushroom” shape are shown in the Figs, this is by way of example only, and any desired number may be utilized).

In another example, the locking mechanism may further comprise member 111 which may be placed through male feature 107 (after outwardly extending projection 107A-C of male feature 107 is locked into undercut 109A in female feature 109) for prohibiting male feature 107 from deflecting (i.e., re-deforming) and allowing outwardly extending projection 107A-C to retract away from undercut 109A in female feature 109 (which retraction could lead to disassembly of tibial spacer 105 from tibial tray 101). In one specific example, member 111 may comprise a threaded fastener (which may be screwed, for example, into corresponding internal threads in male feature 107 and/or corresponding internal threads in female feature 109).

In another example, the locking mechanism may comprise a male feature extending beyond the upper surface of the tibial tray and a female feature disposed in the lower surface of the tibial spacer for receiving the male feature (wherein the male feature is elastically deformable in order to lock at least one outwardly extending projection of the male feature into at least one undercut in the female feature). The locking mechanism may further comprise a member which may be placed through the male feature for prohibiting the male feature from deflecting (i.e., re-deforming) and allowing the outwardly extending projection to retract away from the undercut in the female feature (which retraction could lead to disassembly of the tibial spacer from the tibial tray). In one specific example, the member may comprise a threaded fastener (which may be screwed, for example, into corresponding internal threads in male feature and/or corresponding internal threads in female feature).

In another example, the locking mechanism may comprise male feature 113 extending beyond the lower surface of tibial insert 103 and female feature 115 disposed in the upper surface of tibial spacer 105 for receiving male feature 113 (male feature 113 may be elastically deformable in order to lock at least one outwardly extending projection 113A (forming a “mushroom” shape) of male feature 113 into at least one undercut 115A in female feature 115).

In another example, the locking mechanism may comprise a male feature extending beyond the upper surface of the tibial spacer and a female feature disposed in the lower surface of the tibial insert for receiving the male feature (the male feature may be elastically deformable in order to lock at least one outwardly extending projection of the male feature into at least one undercut in the female feature).

In another example, tibial tray 101 may further comprise upwardly extending sidewall 116 and the locking mechanism may comprises at least one undercut 116A in upwardly extending sidewall 116 and at least one tab 117 on tibial spacer 105 which locks into undercut 115A (in one example, one or both of undercut 116A and/or tab 117 may be elastically deformable).

In another example, the tibial spacer may further comprise a downwardly extending sidewall and the locking mechanism may comprise at least one undercut in the downwardly extending sidewall and at least one tab on the tibial tray which locks into the undercut (in one example, one or both of the undercut and/or the tab may be elastically deformable).

In another example, tibial spacer 105 may further comprise upwardly extending sidewall 119 and the locking mechanism may comprise at least one undercut 119A in upwardly extending sidewall 119 and at least one tab 121 on tibial insert 103 which locks into the undercut (in one example, one or both of undercut 119A and/or tab 121 may be elastically deformable).

In another example, the tibial insert may further comprise a downwardly extending sidewall and the locking mechanism may comprise at least one undercut in the downwardly extending sidewall and at least one tab on the tibial spacer which locks into the undercut (in one example, one or both of the undercut and/or the tab may be elastically deformable).

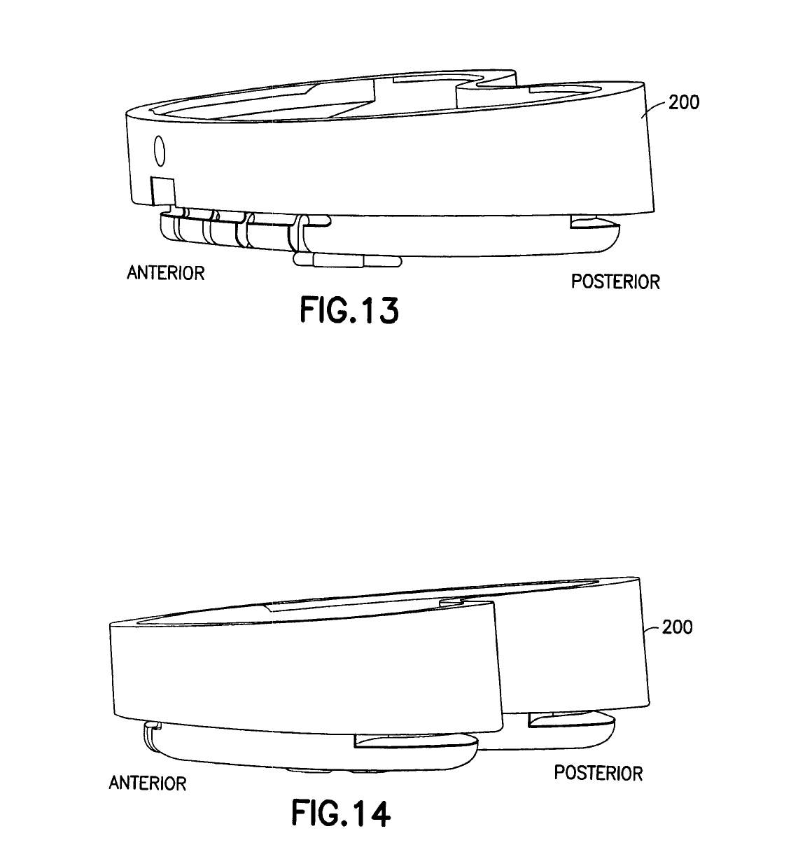

Referring now to FIGS. 13-20, it is seen that in various examples the upper surface and the lower surface of the tibial spacer may be non-parallel to one another (e.g., the upper surface the tibial spacer (that is, the surface adjacent the tibial insert) may provide a varus/valgus correction and/or the upper surface of the tilbial spacer (that is, the surface adjacent the tibial insert) may provide a flexion/extension correction).

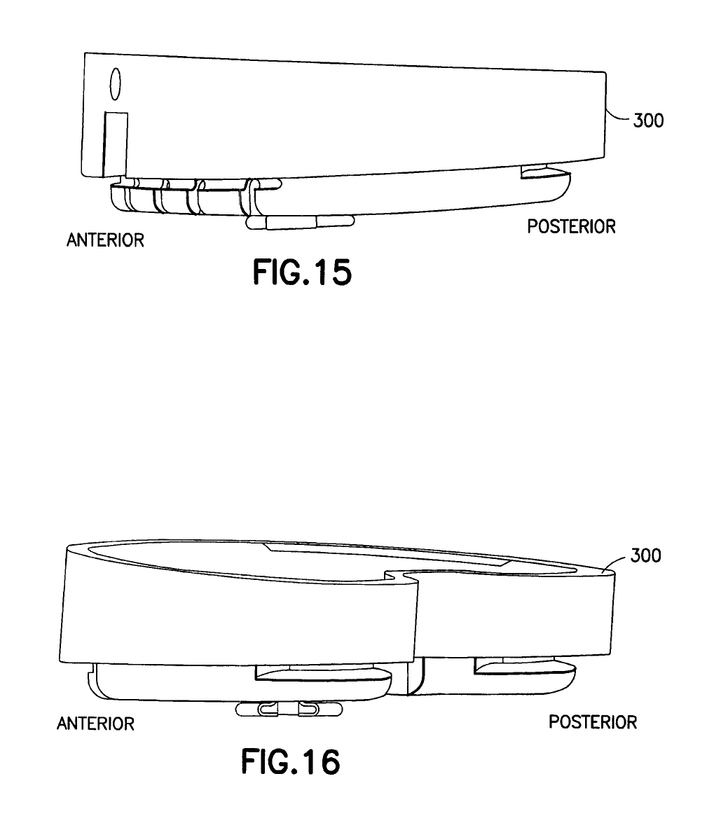

In the specific examples of FIGS. 13-16, it is seen that the upper surface of tibial spacer 200 may slope downwards from the posterior towards the anterior (see FIGS. 13 and 14) or the upper surface of tibial spacer 300 may slope upwards from the posterior towards the anterior (see FIGS. 15 and 16).

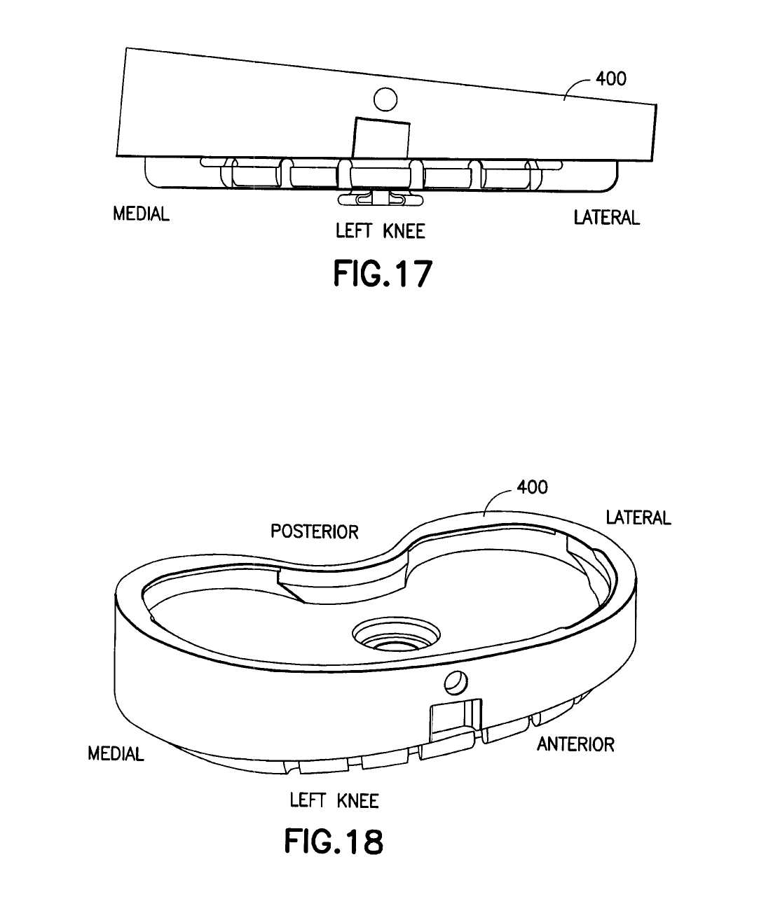

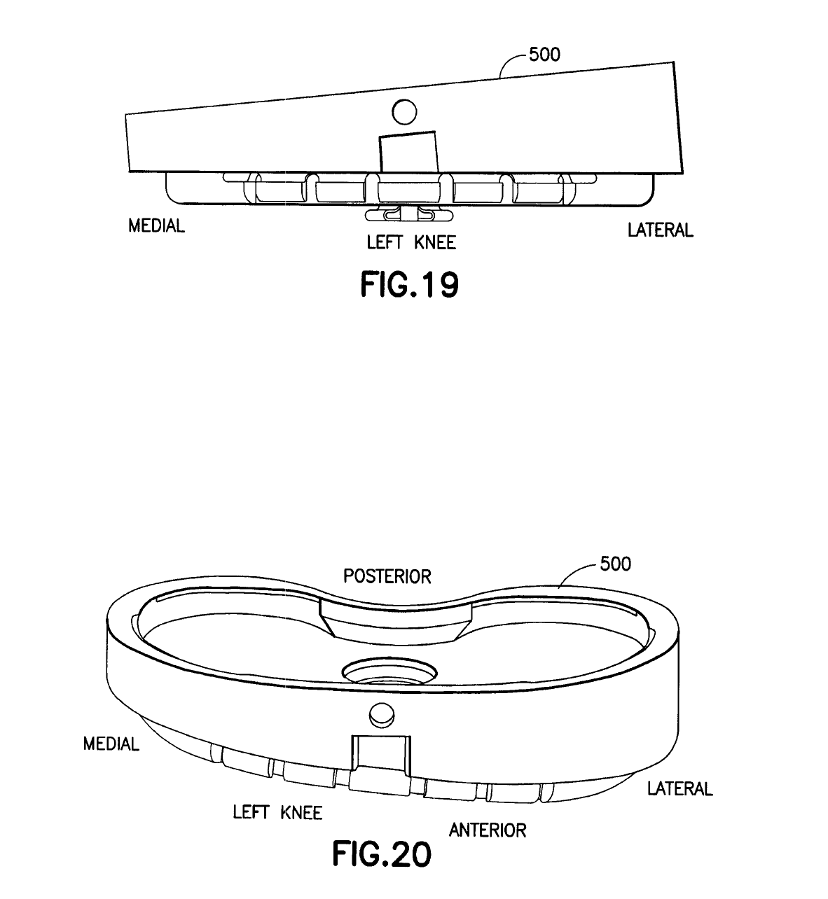

In the specific examples of FIGS. 17-20, it is seen that the upper surface of tibial spacer 400 may slope downwards from the medial towards the lateral (see FIGS. 17 and 18) or the upper surface of tibial spacer 500 may slope upwards from the medial towards the lateral (see FIGS. 19 and 20). Of course, while these FIGS. 17-20 relate to the left knee, corresponding configurations for the right knee may be provided.

In another embodiment, a method for implanting a knee prosthesis in a patient is provided, comprising: providing a tibial tray, wherein the tibial tray comprises an upper surface and a lower surface, and the lower surface of the tibial tray is disposed adjacent a tibia of the patient; providing a tibial insert which has a shelf life and which is not re-sterilizable after the shelf life has expired, wherein the tibial insert comprises an upper surface and a lower surface; and providing a tibial spacer which has a shelf life and which is re-sterilizable after the shelf life has expired, wherein the tibial spacer comprises an upper surface and a lower surface, and wherein the tibial spacer is disposed between the tibial tray and the tibial insert such that the lower surface of the tibial spacer is adjacent the upper surface of the tibial tray and the upper surface of the tibial spacer is adjacent the lower surface of the tibial insert. In one example, the steps may be carried out in the order recited.

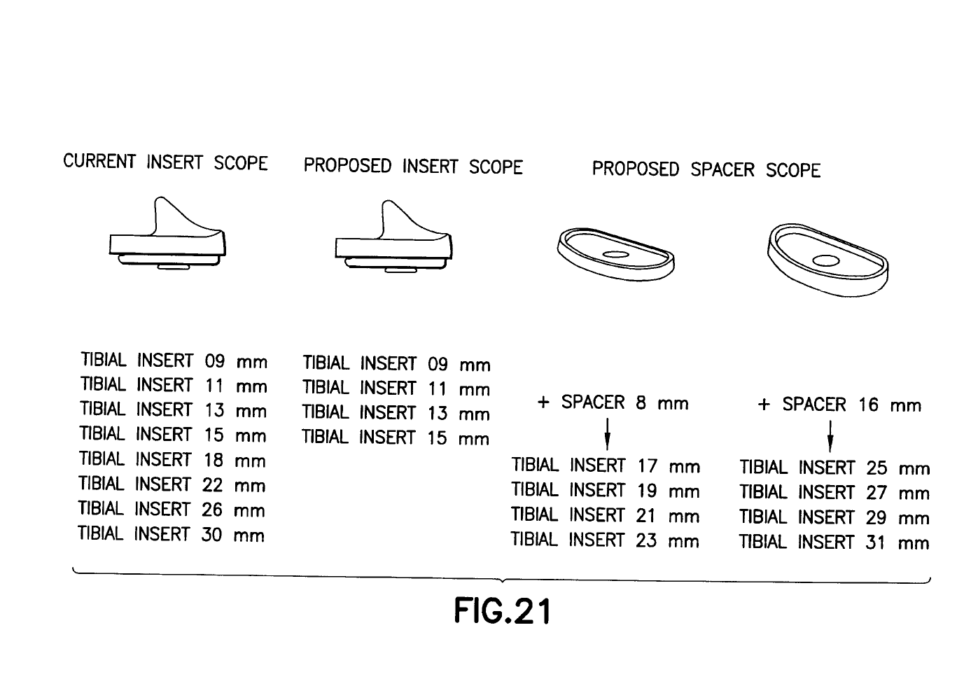

Referring now to FIG. 21, a diagram which highlights some of the benefits of various embodiments of the present invention as applied to a total knee system is shown (for the purposes of this example, the invention is applied to the Exactech Optetrak Posterior Stabilized knee system—of course, the invention may be applied to other systems, as desired). As seen in this FIG. 21, the example component thicknesses are provided to illustrate the concept.

Based on what is shown in FIG. 21, the tibial spacer enables removing the thicker tibial inserts (e.g., greater than 15 mm) from the scope of the total knee system product line. As a result, the surgeon would only use “fresh” UHMWPE tibial inserts (i.e., tibial inserts with a short shelf aging date at the time of surgery), since the UHMWPE tibial insert scope should, in this example, constitute only the most frequently used inserts (e.g., 15 mm or thinner).

In addition, because the proposed scope of this example includes no thick UHMWPE tibial inserts, the number of returned UHMWPE tibial inserts that have exceeded the shelf age date limit should substantially decrease.

In another example, the tibial spacer could also provide an advantage of being available in a “downsized” option. This would provide the surgeon the opportunity to use a tibial tray smaller than the femoral component. This benefit is particularly relevant when the surgeon cuts a large portion of the proximal tibia due to the abrupt decrease of the transverse section of the tibia—in such cases, the tibial tray size could be much smaller than the femoral component size.

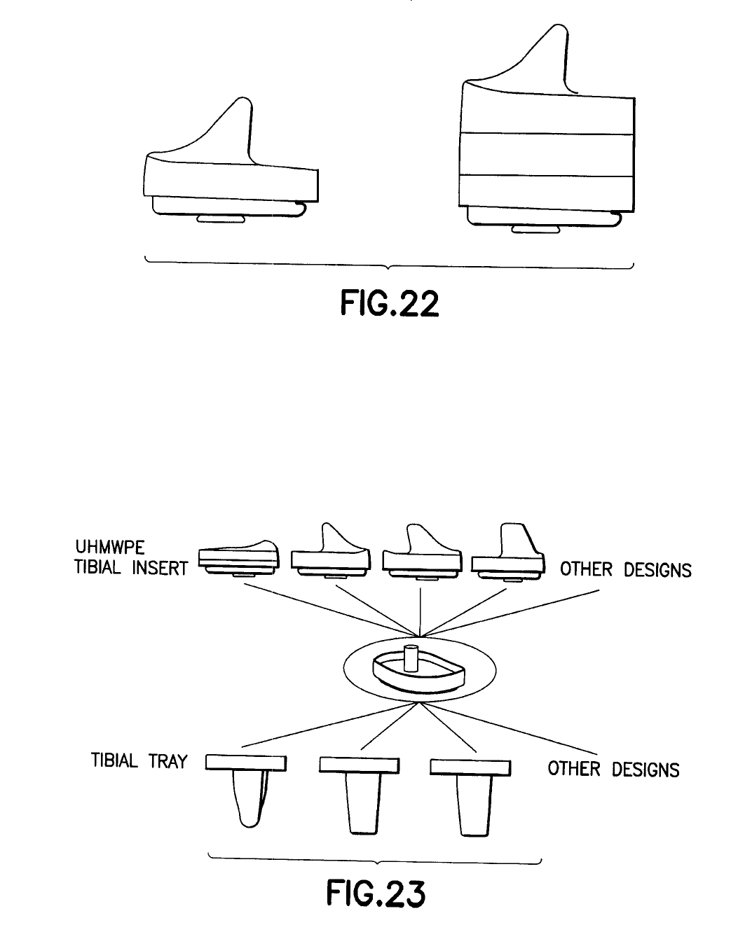

In another example, the use of the tibial spacer could potentially provide less stress on the locking feature (e.g., the mushroom) of the UHMWPE tibial insert. In this regard, as FIG. 22 illustrates, the moment arm of the resultant load increases with the thickness of the UHMWPE tibial insert. As a result, the locking feature of a thick UHMWPE insert (pictured right) is more stressed than the locking feature of a thin UHMWPE insert (pictured left).

In another example, the tibial spacer could be “universal” for the entire range of a total knee system. FIG. 23 illustrates this by applying the tibial spacer to the Exactech Optetrak Comprehensive Total Knee System.

In another example, the tibial spacer may comprise metal.

In another example, the tibial spacer may consist entirely of metal.

In another example, the tibial tray may comprise metal.

In another example, the tibial tray may consist entirely of metal.

In another example, the tibial insert may comprise plastic (e.g., UHMWPE).

In another example, the tibial insert may consist entirely of plastic (e.g., UHMWPE).

In another example, any metal parts may be deformable (e.g., elastically deformable) due to the type of metal and/or due to the geometric configuration (e.g., the metal may be deformable at one or more thinner areas).

In another example, any plastic parts may be deformable (e.g., elastically deformable) due to the type of plastic and/or due to the geometric configuration (e.g., the plastic may be deformable at one or more thinner areas).

In another example, the tibial insert may comprise or consist entirely of a non-UHMWPE (e.g., PEEK).

In another example, the present invention may be applied to a fixed bearing knee prosthesis.

In another example, the present invention may be applied to a mobile bearing knee prosthesis or a rotating bearing knee prosthesis (e.g., where the tibial insert is allowed to move anterior/posterior relative to the tibial spacer/tray, where the tibial insert is allowed to move medial/lateral relative to the tibial spacer/tray, and/or where the tibial insert is allowed to rotate relative to the tibial spacer/tray).

In another example, locking of the tibial spacer to the tibial tray and/or to the tibial insert may prohibit (or limit) relative up/down movement and/or may prohibit (or limit) relative movement in any one or more other possible degrees of freedom (e.g., anterior/posterior, medial/lateral).

In another example, the upper surface of the tibial spacer may be essentially the same shape as (e.g., equivalent to) the upper surface of the tibial tray and/or the lower surface of the tibial spacer may be essentially the same shape as (e.g., equivalent to) the lower surface of the tibial insert (e.g., for cross compatibility).

While a number of embodiments of the present invention have been described, it is understood that these embodiments are illustrative only, and not restrictive, and that many modifications may become apparent to those of ordinary skill in the art. For example, any element described herein may be provided in any desired size (e.g., any element described herein may be provided in any desired custom size or any element described herein may be provided in any desired size selected from a “family” of sizes, such as small, medium, large). Further, one or more of the components may be made from any of the following materials: (a) any biocompatible material (which biocompatible material may be treated to permit surface bone ingrowth or prohibit surface bone ingrowth—depending upon the desire of the surgeon); (b) a plastic; (c) a fiber; (d) a polymer; (e) a metal (a pure metal such as titanium and/or an alloy such as Ti—Al—Nb, Ti-6Al-4V, stainless steel); (f) any combination thereof. Further still, any metal construct may be a machined metal construct. Further still, any number of protrusions (e.g., such as for initial fixation by forming a bond with cement and/or such as for supplemental fixation by forming a bond with cement) may be utilized with a given prosthesis. Further still, any number of female features that increase the cement mantle may be utilized with a given prosthesis. Further still, any number of male features that could dig into the bone so that initial/supplemental fixation can be improved may be utilized with a given prosthesis. Further still, any number of bone screws (e.g., such as for initial fixation and/or such as for supplemental fixation) may be utilized with a given prosthesis. Further still, any steps described herein may be carried out in any desired order (and any additional steps may be added as desired and/or any steps may be deleted as desired).

1. A knee prosthesis for implantation in a patient, comprising:
a tibial tray, wherein the tibial tray comprises an upper surface and a lower surface, and the lower surface of the tibial tray is disposed adjacent a tibia of the patient;
a tibial insert, wherein the tibial insert comprises an upper surface and a lower surface;
a tibial spacer, wherein the tibial spacer comprises an upper surface and a lower surface, and wherein the tibial spacer is disposed between the tibial tray and the tibial insert such that the lower surface of the tibial spacer is adjacent the upper surface of the tibial tray and the upper surface of the tibial spacer is adjacent the lower surface of the tibial insert; and
at least one locking mechanism, wherein the locking mechanism locks the tibial spacer to the tibial tray to form a locked spacer/tray assembly such that the locking mechanism substantially prohibits at least relative up-down movement between the tibial spacer and the tibial tray when the locked spacer/tray assembly is implanted in the patient.

2. The prosthesis of claim 1, wherein the locked spacer/tray assembly can carry joint loads when implanted in the patient.

3. The prosthesis of claim 2, wherein the locked spacer/tray assembly allows, when implanted in the patient, ambulation and activities of daily living.

4. The prosthesis of claim 1, wherein the upper surface of the tibial insert is configured to receive a femoral component which interfaces with a femur of the patient.

5. The prosthesis of claim 1, wherein the upper surface and the lower surface of the tibial spacer are essentially parallel to one another.

6. The prosthesis of claim 1, wherein the upper surface and the lower surface of the tibial spacer are non-parallel to one another.

7. The prosthesis of claim 6, wherein the upper surface of the tibial spacer provides a varus/valgus correction.

8. The prosthesis of claim 6, wherein the upper surface of the tibial spacer provides a flexion/extension correction.

9. The prosthesis of claim 1, wherein the upper surface of the tibial spacer is essentially the same shape as the upper surface of the tibial tray.

10. The prosthesis of claim 1, wherein the lower surface of the tibial spacer is essentially the same shape as the lower surface of the tibial insert.

11. The prosthesis of claim 1, wherein the upper surface of the tibial spacer is essentially the same shape as the upper surface of the tibial tray and the lower surface of the tibial spacer is essentially the same shape as the lower surface of the tibial insert.

12. The prosthesis of claim 1, wherein the locking mechanism comprises a male feature extending beyond the lower surface of the tibial spacer and a female feature disposed in the upper surface of the tibial tray for receiving the male feature, and wherein the male feature is deformable in order to lock at least one outwardly extending projection of the male feature into at least one undercut in the female feature.

13. The prosthesis of claim 12, wherein the locking mechanism further comprises a member extending through the male feature for prohibiting the male feature from re-deforming and allowing the outwardly extending projection to retract away from the undercut in the female feature after the outwardly extending projection has been locked into the undercut in the female feature.

14. The prosthesis of claim 13, wherein the member comprises a threaded fastener.

15. The prosthesis of claim 1, wherein the locking mechanism comprises a male feature extending beyond the upper surface of the tibial tray and a female feature disposed in the lower surface of the tibial spacer for receiving the male feature, and wherein the male feature is deformable in order to lock at least one outwardly extending projection of the male feature into at least one undercut in the female feature.

16. The prosthesis of claim 15, wherein the locking mechanism further comprises a member extending through the male feature for prohibiting the male feature from re-deforming and allowing the outwardly extending projection to retract away from the undercut in the female feature after the outwardly extending projection has been locked into the undercut in the female feature.

17. The prosthesis of claim 16, wherein the member comprises a threaded fastener.

18-31. (canceled)

32. A method for implanting a knee prosthesis in a patient, comprising:
providing a tibial tray, wherein the tibial tray comprises an upper surface and a lower surface, and the lower surface of the tibial tray is disposed adjacent a tibia of the patient;
providing a tibial insert which has a shelf life and which is not readily re-sterilizable after the shelf life has expired, wherein the tibial insert comprises an upper surface and a lower surface; and
providing a tibial spacer which has a shelf life and which is re-sterilizable after the shelf life has expired, wherein the tibial spacer comprises an upper surface and a lower surface, and wherein the tibial spacer is disposed between the tibial tray and the tibial insert such that the lower surface of the tibial spacer is adjacent the upper surface of the tibial tray and the upper surface of the tibial spacer is adjacent the lower surface of the tibial insert.

33. The method of claim 32, further comprising locking the tibial spacer to the tibial tray to form a locked assembly such that the locking mechanism substantially prohibits at least relative up-down movement between the tibial spacer and the tibial tray when the locked assembly is implanted in the patient.

34. The method of claim 33, wherein the locked assembly can carry joint loads when implanted in the patient.

35. The method of claim 34, wherein the locked assembly allows, when implanted in the patient, ambulation and activities of daily living.

36. The method of claim 32, wherein the upper surface of the tibial insert is configured to receive a femoral component which interfaces with a femur of the patient.

37. The method of claim 32, wherein the steps are carried out in the order recited.
